(19) United States
(12) Patent Application Publication
(10) Pub. No.: US 2019/0164636 A1
(43) Pub. Date: May 30, 2019

(54) NATIONAL PATIENT PHARMACY IDENTIFIER

(71) Applicant: Ronald Lynn Power, Hilton Head, SC (US)

(72) Inventor: Ronald Lynn Power, Hilton Head, SC (US)

(21) Appl. No.: 16/175,767

(22) Filed: Oct. 30, 2018

Related U.S. Application Data
(63) Continuation of application No. 15/943,345, filed on Apr. 2, 2018, now abandoned.
Application No. 15/943,345 is a continuation of application No. 14/619,817, filed on Feb. 11, 2015, now abandoned.
(60) Provisional application No. 62/020,548, filed on Jul. 3, 2014.

(51) Int. Cl. G16H 20/10; G06F 16/335; G16H 70/40

(52) U.S. Cl. CPC G16H 20/10; G16H 70/40; G06F 16/337

(57) ABSTRACT

Social Prescription Identification Number (SPIN) is a unique national outpatient identification number to collectively and individually identify every pharmacy patient in one national interoperable super database. The super database will interface with America's interdependent, competitively fragmented retail pharmacy networks, tracking individual patient prescription transactions. SPIN will function as an insurance identification number offering comprehensive necessary real time prescription adjudication information from insurance providers. One national pharmacy identification number in its own national database, is an option offering U.S. government and/or American retail pharmacy, benefits of a national provider outpatient prescription service overlay of a diverse retail pharmacy network. Technological evolution, configuration change, and national pharmacy network diversity may not be conducive to cellular modules during patent formation.

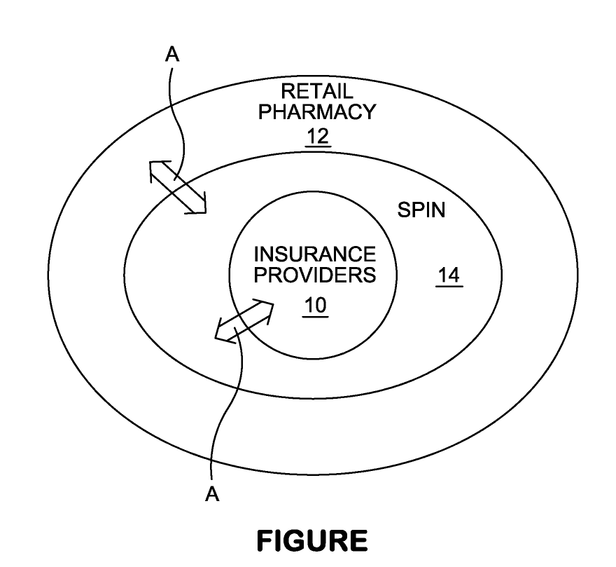

### REFERENCE TO RELATED CASES

The present patent application is based upon and hereby claims priority to U.S. Provisional Patent Application Ser. No. 62/020,548 filed Jul. 3, 2014, entitled “National Outpatient Pharmacy Identifier:” and the disclosure of that provisional patent application is hereby incorporated herein in its entirety by reference.

### BACKGROUND OF THE INVENTION

Controlled substance prescription overdose deaths have increased more in the U.S. since 1990 than in European Union Member Nations. The European Union Member Nations have a population close to 500 million people with the United States population close to 300 million people. Since a large portion of the U.S. population is of European descent, similar genetic variation (phenotypic characteristics) would be expected pharmacologically with prescription drugs. The classes of prescription drugs used between the two populations is comprised of similar entities with the same mechanisms of action. Physicians from both groups share the same medical diversity, integrity, medical science, medical procedures, and standards of care. Parameters point to all being equal; pharmacogenomics, drugs, and physicians. Yet in 2011, there were less than 7,000 prescription overdose deaths in European Union Member Nations and greater than 40,000 prescription overdose deaths in the U.S. One variable is prescription delivery systems.

American retail pharmacies compete by increasing amounts of wellness offered to patients and potential patients. However, current configuration of network delivery enables exposure of unintended prescription controlled substances to the general population. Retail pharmacy best practices and future electronic, technological advances may not be sufficient to bridge current system breaches. To date, each commercial pharmacy has its own databases and they do not communicate with each other, thereby leaving open the possibility that a patient could duplicate a prescription at different commercial pharmacies or can extend the length of a particular pharmaceutical drug beyond the time prescribed by a physician.

In addition, there are currently databases set up in almost all States, however, again there is not any complete cross-communication between the States such that a patient can have a prescription filed in one State and then cross the border into another State and duplicate the prescription since there is no single overall entity that receives the information from differing States recognizing duplications.

Non-interfacing networks or systems lacking structural interoperability necessitate pharmacists to leave individual network workflow to view offline sources for complete patient prescription transaction information. When offline sources aren't complete and/or pharmacists can't leave workflow, functional deficits or holes occur.

Patients can bring in two to three schedule III to V prescriptions when attending pharmacists are overloaded in their own network workflow. These prescriptions will likely not be viewed in a State Prescription Drug Monitoring Program network. If that same patient has already had a CII Rx filled in a separate pharmacy database written by a different physician, prescription surveillance will be negated. Since pharmacists have heightened clinical efficiency in their own workflow, functionality permitting complete singular view of patient prescription records without leaving workflow is necessary. It would create a significant, positive impact on drug diversion and overdose deaths.

It would thus be advantageous to have all pharmacy transactions throughout the U.S. comprise a national outpatient prescription delivery system that necessitates patient registration into one super database, one time, with their unique number, the Social Prescription Identification Number, hereinafter referred to as SPIN populating their individual prescription insurance identification number field. The SPIN super database would interface real time with every database network bringing total prescription transactions per patient to each pharmacist in workflow, keeping pharmacists in workflow. Complete real-time prescription information would be available to emergency room physicians, medical center or clinic physicians, hospital admissions intake, a physician during office visits, provider monitoring, and even during an emergency with an unconscious patient.

### SUMMARY OF THE INVENTION

Prescription overdose deaths in the U.S. have exceeded 200,000 people in the past 14 years. Recorded statistics may be extremely conservative with death certificates not necessarily relating the actual cause of death. The number of individuals that habitually abuse illicit controlled substance prescriptions has exceeded one million people and may be approaching two million people. Habitual controlled substance abuse creates subconscious hardwiring of the mesolimbic midbrain region. The human midbrain is architected with four different centers providing survival instincts:

Hippocampus—tissue that acts as a memory indexer (stored, organized and retrieved)

Amygdala—tastes incoming information in brain for emotional content (fear, pleasure, surprise and etc)

Ventral Tegmental Area (VTA)—sorts and coordinates information from other areas of brain for satisfaction. Forwards good readings by amount of dopamine released.

Nucleus Accumbens—site of where dopamine content regulates motivation for food and other pleasurable activities.

When prescription controlled substances are abused for more than three months, new neurotransmitter pathways are formed through these centers and end in the prefrontal cortex (conscious) or executive center of the human brain. Portions of dopamine release become dependent on these new pathways and necessitate drugs of abuse for activity. Opioid and other narcotic drug abuse patients have extremely low addiction recovery rates, some sources list less than ten percent. Consequently, one to two million people in our nation will bear degrees of lifelong dependency.

America has 2,500 youth (12-17 years) abusing a prescription pain reliever for the first time every day. Since 200,000 retail pharmacists are not capable of monitoring multiple networks (electronic data silos) simultaneously, interoperability bringing complete patient prescription transactions real time into their own network workflow, will significantly impact death rates and quality of life.

As such, in order to prevent the overdosing of patients and to make sure that prescriptions are not filled more than once, and accurately, there is herein provided, a system and method that insures that any individual patient cannot get a prescription filled in more than one location, if not deemed necessary, even if it is across State lines. Each individual patient is given a universal, nation-wide number, that is, a SPIN that is a unique national outpatient identification number to collectively and individually identify every pharmacy patient in one national interoperable super database. A pharmacy provider can thus conduct an accurate and comprehensive search under a patient SPIN and immediately glean useful information, for example, information as to whether the patient has been overusing a drug and refer that patient to a physician to determine if the prescription is legitimate or needs further scrutiny.

The SPIN is administered and stored in a super database that will interface with America's interdependent, competitively fragmented retail pharmacy networks, tracking individual patient prescription transactions. SPIN will function as an insurance identification number offering comprehensive necessary real time prescription adjudication information from insurance providers. One national pharmacy identification number in its own national database is an option offering U.S. government and/or American retail pharmacy, benefits of a national provider outpatient prescription service overlay of a diverse retail pharmacy network.

Other objects and advantages will become apparent to those skilled in the art from a review of the ensuing detailed description which proceeds from the following illustrative drawings.

### BRIEF DESCRIPTION OF THE DRAWINGS

The FIGURE is a schematic view illustrating the flow of information to and from the super database issuing and maintaining the SPIN and the insurance providers and the retail pharmacies.

### DESCRIPTION OF A PREFERRED EMBODIMENT

Turning to the FIGURE, there is a schematic view of the system and method of the present invention to illustrate the flow of information and data. As can be seen, the center circle 10 is representative of the insurance providers while the outer ring 12 is representative of the retail pharmacies. In the intermediate ring, there is the super database 14 that allocates the SPIN's and both receives and provides information from both the insurance providers and the retail pharmacies. The direction of the flow of information is depicted by the double arrows A.

An outline of the present invention is initially instructive in order to understand the flow of information and data depicted in the FIGURE. As a first step, the patient must obtain a SPIN, that is, a number that is personal to each individual and one that is protected from duplication or confusion as to any other individual. The SPIN is issued to an individual based on certain information for that individual patient that assures that the correct person is being supplied and identified with the SPIN, that is, the information may include physical and e-mail address, age, date of birth, gender, telephone numbers, parent/spouse, allergies, military ID, drivers license number, birth certificate, passport and demographic data or, preferable, biometric data such as a fingerprint, iris scan, DNA sample or the like. It is important that there be sufficient information and data to identify the patient with certainty so that the SPIN can be used with assurance that the correct patient is being treated.

While much of the data and information used to identify a patient include various numerical values, it is important that the individual information not include a social security number or any financial identifier to prevent the dissemination of that type of information that can be used for nefarious purposes.

In any event, the SPIN is then issued after proper and certain identification of the patient and the SPIN, once issued, stays with the individual patient for life. The patient may apply on line to obtain the SPIN, thereby accessing the super database and will be prompted to supply certain of the aforementioned personal information or data. The SPIN can be embodied in a ID card that the patient can carry that includes protective identifiers, such as a picture ID or other means that allows the pharmacist to make a positive identification of the patient.

As such, the SPIN is available in the super database 14 and information and data is therefore stored in the super database 14 to be accessed using the SPIN.

Other information is also inputted into the super database 14 to be available to the retail or other pharmacy or to an insurance provider.

For example, a pharmacy supplies information and data to the super database 14 indicative of a particular transaction and, in particular, can include the personal patient SPIN, pharmacy provider identifier, pharmacy location and store number, physician provider identifier, drug identifiers, drug quantity and days of supply, prescription written dates, prescription fill dates, prescription new or refill status and prescription number. That information and data is provided as input to the super database 14 no matter whether the pharmacy is a Walgreen's, CVS, hospital pharmacy or other pharmacy so that the super database 14 can amass and store that information that is relative to further prescriptions for the patient having that SPIN.

Also inputted by the pharmacy can be, but is not limited to, the drug identifiers, the national drug code, strength, form, quantity, lot number and expiration date. As such, in the event there is a recall of any particular drug, all of the pertinent information concerning that drug, including patients, concerned pharmacies, and the like, can be immediately available in the super database 14 and the appropriate parties can be quickly notified by e-mail or other rapid information transfer.

The insurance provider also inputs information and data into the super database 14, such as the patient's necessary insurance routing information and billing information so that such information is available to the prescription network to aid in allocating the proper insurance coverage for each patient. The SPIN will thereafter become the identifier by the insurance provider for that patient

As can now be seen, the advantages of having the super database 14 and the information and data stored therein includes the ability of the system to identify any overuse of any particular medication as well as determine if there are any risks of interaction between drugs being taken by any patient. A halt can immediately be put on the patient for the further purchase of a drug in the event there is evidence of multiple physician prescribing, unusual drug combinations, drug over utilization, multiple pharmacy shopping or level one drug interactions.

In addition, the information and data in the super database 14 is available to and can therefore be accessed by all insurance providers as well as all pharmacies, including commercial pharmacies, VA pharmacies, military pharmacies, hospital emergency room physicians, hospital admission staffs, private physicians, pharmacists working in their own network, emergency medical personal and individual insurance providers. In addition, access can be had by all providers as to drug-drug interaction monitoring between polypharmacy patients and cash paying patients.

The end result is that no patient can have a prescription filled without a SPIN and the pharmacy can look up, in real time, the information and data stored in the super database under the SPIN and glean all of the relevant information to that patient. No patient can have access to a prescription without a SPIN, however, a patient may be allowed a certain limited period of time for a prescription, such as 3-4 days of a non-controlled substance without a SPIN in order to provide the patient sufficient time to obtain a SPIN.

While the present invention has been set forth in terms of a specific embodiment or embodiments, it will be understood that the national patient pharmacy system and method disclosed may be modified or altered by those skilled in the art to other configurations. Accordingly, the invention is to be broadly construed and limited only by the scope and spirit of the claims appended hereto.

What is claimed:

1. A national system for the providing of pharmacy drugs to patients from a plurality of pharmacy providers, the system comprising:
an interoperable super database having a storage medium;
a communications network interface wherein each individual patient can apply for and receive an identification number (SPIN) from the super database based on data of the patient that is unique to the patient and not duplicative;
a communications network interface wherein insurance providers input all necessary patient insurance routing information, adjudication and billing information to the super database for each patient identified by a SPIN as their insurance identification number;
a communication network wherein pharmacy providers can input adjudication, and billing information as well as all necessary prescription post transaction information from a plurality of interdependent outpatient pharmacy providers in the U.S as well as pharmacy provider identifiers, pharmacy location and store number, physician provider identifiers, drug identifiers, drug quantity and days of supply, prescription written date, prescription fill date, prescription new or refill status, and prescription number to the super database;
a communications network wherein pharmacy providers can access the super database to receive necessary insurance routing, adjudication, and billing information as well as all necessary prescription post transaction information from outpatient pharmacy networks in the U.S. consisting of personal patient SPINS, pharmacy provider identifiers, pharmacy location and store number, physician provider identifiers, drug identifiers, drug quantity and days of supply, prescription written date, prescription fill date, prescription new or refill status, and prescription number;
wherein all of the information stored in the super database is accessed by a pharmacy provider prior to providing a pharmacy drug to a patient.

2. The system of claim 1 wherein the data of the patient used to obtain a SPIN includes forms of identification including biometrics and/or state driver's license number, birth certificate, military identification, passport, and demographic data.

3. The system of claim 1 wherein patient data includes SPIN, physical and email address, age, date of birth, gender, telephone numbers, parent/spouse, and allergies.

4. The system of claim 1 wherein drug identifiers include the name, national drug code, strength, form, quantity, lot number and expiration date.

5. The system of claim 1 wherein every individual patient is required by law to have a SPIN prior to being dispensed a drug from a pharmacy provider.

6. The system of claim 1 wherein the SPIN is indicative of data of the patient other than financial information or social security number.

7. A method of providing and controlling the dispensing of a pharmaceutical to a patient from a pharmacy comprising the steps of:
providing an interoperable super database having a storage medium;
inputting data received from a patient into the super database and issuing a SPIN to that patient from that super database that is unique to the patient and not duplicative;
inputting information and data from an insurance provider including all necessary patient insurance routing information, adjudication and billing information to the super database for each patient identified by a SPIN;
inputting data and information from a pharmacy provider relative to all necessary prescription post transaction information from outpatient pharmacy providers in the U.S as well as pharmacy provider identifiers, pharmacy location and store number, physician provider identifiers, drug identifiers, drug quantity and days of supply, prescription written date, prescription fill date, prescription new or refill status, and prescription number to the super database;
providing access to the information and data stored in the super database to pharmacy providers to receive necessary insurance routing, adjudication, and billing information as well as all necessary prescription post transaction information from outpatient pharmacy networks in the U.S. consisting of personal patient identifiers, pharmacy provider identifiers, pharmacy location and store number, physician provider identifiers, drug identifiers, drug quantity and days of supply, prescription written date, prescription fill date, prescription new or refill status, and prescription number;
accessing all of the relevant information for the super database inputted by the insurance providers and the pharmacy providers relevant to the SPIN of a patent to determining how to fill a prescription for that patient.

8. The method of claim 7 further including the step of requiring every pharmacy patient to obtain a SPIN.

9. The method of claim 7 wherein the step of issuing a SPIN comprises issuing a SPIN on the basis of information of the patient other than social security or financial information.

10. The method of claim 7 further including the step of providing to insurance providers complete patient prescription transaction information and other necessary patient information including every patient's SPIN from the super database.

11. The method of claim 7 wherein the step of inputting information and data from an insurance provider comprises inputting information and data using a patient's SPIN as a patient's insurance identification number.

12. The method of claim 7 further including the step of pharmacy providers accessing and using the patient's SPIN located in the super database as the patient's default identification number when there is no prescription insurance.

13. The method of claim 7 wherein the step of accessing all of the relevant information inputted by the insurance providers and the pharmacy providers comprises accessing complete prescription history and necessary transaction information of every pharmacy outpatient available to every insurance provider and outpatient pharmacy network within a predetermined period of time.

14. The method of claim 7 wherein the step of accessing all of the relevant information inputted by the insurance providers and the pharmacy providers comprises accessing, in real time, actual patient drug usage to medical clinics, emergency room physicians, hospital admission staffs, private physician's, pharmacists working in their own network workflow, emergency medical personal, and insurance providers.

15. The method of claim 7 further including the step of providing halts that are programmed into the super database that halt filling a prescription in the event of multiple physician prescribing, unusual drug combinations, drug over utilization, multiple pharmacy shopping, or level one drug interactions.

16. The method of claim 7, wherein the step of providing access to the information and data stored in the super database to pharmacy providers comprises providing access to all providers as to drug-drug interaction monitoring between polypharmacy patients, patients using Veterans Administration pharmacies, military pharmacies, and cash paying patients.

17. The method of claim 7, wherein the step of inputting data received from a patient to insure patient uniqueness and duplication prevention include patient identification including biometrics and/or state driver's license number, birth certificate, military identification, passport, and demographic data.

18. The method of claim 7 wherein the step of inputting data received from a patient include, physical and/or email address, age, date of birth, gender, telephone numbers, parent/spouse, and allergies.

19. The method of claim 7 wherein the step of inputting data and information from a pharmacy provider of drug identifiers includes inputting the national drug code, strength, form, quantity, lot number and expiration date.
